(19) United States
(12) Patent Application Publication
(10) Pub. No.: US 2019/0137476 A1
(43) Pub. Date: May 9, 2019

(54) DETECTING HUANGLONGBING (HLB) IN CITRUS PLANTS BY ANALYZING CHANGES IN EMITTED VOLATILE ORGANIC COMPOUNDS

(71) Applicant: The Regents of the University of California, Oakland, CA (US)

(72) Inventors: Cristina E. Davis, Davis, CA (US); Abhaya M. Dandekar, Davis, CA (US); Alexander A. Aksenov, San Diego, CA (US); Alberto Pasamontes Funez, Tarragona (ES); Daniel J. Peirano, Davis, CA (US); Mitchell M. McCartney, Davis, CA (US); Oliver Fiehn, Davis, CA (US); Susan E. Ebeler, Davis, CA (US); Yuriy Zrodnikov, West Sacramento, CA (US)

(73) Assignee: The Regents of the University of California, Oakland, CA (US)

(21) Appl. No.: 16/086,665

(22) PCT Filed: Mar. 23, 2017
(86) PCT No.: PCT/US17/23909

§ 371 Date: Sep. 20, 2018

Related U.S. Application Data
(60) Provisional application No. 62/313,501, filed on Mar. 25, 2016.
(60) Provisional application No. 62/313,435, filed on Mar. 25, 2016.

(51) Int. Cl. G01N 33/487; B01L 3/00; B01D 53/04; B01D 53/02

(52) U.S. Cl. CPC G01N 33/48707; B01L 3/508; B01D 53/0415; B01D 53/025; B01D 2257/708; B01L 2300/069; B01L 2300/047; B01L 2200/0689; B01D 2253/202; B01L 2300/123

(57) ABSTRACT

The disclosed embodiments relate to a technique for detecting Huanglongbing (HLB) infection in a citrus plant. This technique involves first gathering one or more samples of volatile organic compounds (VOCs) emanating from the citrus plant. Next, a system measures VOCs in the gathered samples to determine a VOC profile for the citrus plant, wherein the VOC profile comprises measured values for a set of VOCs that comprise disease-specific biomarkers for HLB infection. Finally, the system determines an HLB infection status for the citrus plant by analyzing the VOC profile.

This application claims priority under 35 U.S.C. § 119 to the following U.S. provisional patent applications: Application No. 62/313,501, filed 25 Mar. 2016 (Atty. Docket No.: UC16-439-1PSP); and Application No. 62/313,435, filed 25 Mar. 2016 (Atty. Docket No.: UC16-444-1PSP). The contents of the above-listed applications are incorporated by reference herein in their entirety.

### BACKGROUND

### Field

The disclosed embodiments generally relate to techniques for detecting diseases in plants. More specifically, the disclosed embodiments relate to techniques for detecting HLB in citrus plants by analyzing emitted volatile organic compounds (VOCs).

### Related Art

The vector-borne plant disease “citrus greening” or Huanglongbing (HLB), which is caused by the bacterium Candidatus Liberibacter asiaticus and spread by the phloem-feeding insect Asian citrus psyllid, is presently afflicting citrus crops worldwide. Although not harmful to human health, HLB is devastating to citrus plants due to its effect on production, tree decline, fruit size and fruit shape. Because citrus plants remain asymptomatic for the disease over long periods, it is important to identify the infection before symptoms appear. If detected at an early stage, transmission of the disease from infected trees can be halted or diminished via selective tree removal. Infected trees can also be provided with elevated nutrient therapy to minimize symptoms and reduce disease development as well. To date, there exist few techniques that are able to detect citrus infection with the Candidatus Liberibacter bacterium when it is asymptomatic. Polymerase chain reaction (PCR) testing is one potential technique for diagnosing HLB. However, PCR is an expensive and time-consuming process, which is further challenging because the bacterial loads in plants are distributed unevenly and can fluctuate with time.

Hence, what is needed is a new technique for detecting infection of a citrus plant with the Candidatus Liberibacter bacterium while the citrus plant is asymptomatic without the above-described drawbacks of existing techniques.

### SUMMARY

The disclosed embodiments relate to a technique for detecting Huanglongbing (HLB) infection in a citrus plant. This technique involves first gathering one or more samples of volatile organic compounds (VOCs) emanating from the citrus plant. Next, a system measures the VOCs in the gathered samples to determine a VOC profile for the citrus plant, wherein the VOC profile comprises measured values for a set of VOCs that comprise disease-specific biomarkers for HLB infection. Finally, the system determines an HLB infection status for the citrus plant by analyzing the VOC profile.

In some embodiments, gathering the samples of the VOCs involves in situ collection of the samples.

In some embodiments, the in situ collection of the sample involves using a sorbent-based sampling methodology.

In some embodiments, the sorbent-based sampling methodology involves using a polydimethylsiloxane (PDMS)-based absorptive bead.

In some embodiments, gathering the samples involves gathering each sample for a predetermined duration spanning minutes to hours.

In some embodiments, gathering the samples involves gathering the samples at specific times of day.

In some embodiments, measuring the VOCs in the gathered samples involves using gas chromatography and/or mass spectrometry (GC/MS) to perform the measurements.

In some embodiments, while determining the HLB infection status for the citrus plant, the system applies a statistical model, such as partial least squares discriminant analysis (PLS-DA), to the VOC profile to determine probability values for each possible HLB infection status. Next, the system determines the HLB infection status for the citrus plant based on the determined probability values.

In some embodiments, applying statistical model to the VOC profile involves multiplying the measured value for each disease-specific biomarker in the VOC profile with a corresponding coefficient obtained from one or more tables of coefficients for the disease-specific biomarkers.

In some embodiments, the one or more tables of coefficients account for one or more of: season-specific alterations, varietal alterations, and geographic alterations of the disease-specific biomarkers.

In some embodiments, the one or more tables of coefficients are stored in a database.

In some embodiments, the HLB infection status for the plant comprises at least one of: healthy; infected asymptomatic; mildly infected symptomatic; and severely symptomatic.

The disclosed embodiments relate to a device that facilitates handling a sorbent bead to facilitate using the sorbent bead to sample chemical compounds. The device includes a storage enclosure for holding the sorbent bead, wherein the storage enclosure is sealable to prevent contamination of the sorbent bead during transport and storage. It also includes a sampling enclosure for holding the sorbent bead, wherein the sampling enclosure is perforated to allow chemical compounds to come into contact with the sorbent bead while the sorbent bead is being used to sample the chemical compounds. It additionally includes a sealable interface between the storage enclosure and the sampling enclosure. When unsealed, the sealable interface provides an opening to facilitate moving the sorbent bead between the storage enclosure and the sampling enclosure without physical handling of the sorbent bead by a user.

In some embodiments, the sampling enclosure comprises a chemically inert mesh.

In some embodiments, the storage enclosure comprises a sealable vial.

In some embodiments, the storage enclosure is detachable from the device.

In some embodiments, the sampling enclosure is detachable from the device.

In some embodiments, the device further comprises a suspension mechanism for suspending the device at a sampling location while the sorbent bead is held in the sampling enclosure.

In some embodiments, the sorbent bead comprises a stir bar sorptive extraction (SBSE) bead.

In some embodiments, the device includes a tracking mechanism.

In some embodiments, the tracking mechanism comprises a label, such as a barcode or a radio-frequency identification (RFID) tag.

In some embodiments, the tracking mechanism comprises a global-positioning system (GPS) tag.

### BRIEF DESCRIPTION OF THE FIGURES

FIG. 1 illustrates a chemical-analysis system in accordance with the disclosed embodiments.

FIG. 2 presents a flowchart illustrating how the chemical-analysis system operates in accordance with an embodiment of the present disclosure.

FIGS. 3A-3F collectively comprise a table containing season-specific fall/winter coefficients for determination of HLB infection health status in accordance with the disclosed embodiments.

FIGS. 4A-4B collectively comprise a table containing season-specific summer/fall coefficients for determination of HLB infection health status in accordance with the disclosed embodiments.

FIGS. 5A-5E collectively comprise a table containing season-specific winter/spring coefficients for determination of HLB infection health status in accordance with the disclosed embodiments.

FIGS. 6A-6I collectively comprise a table containing coefficients for determination of HLB infection health status in accordance with the disclosed embodiments.

FIG. 7A illustrates an SBSE-bead protection apparatus with a threaded attachment in accordance with an embodiment of the present disclosure.

FIG. 7B illustrates an SBSE-bead protection apparatus with a clamped attachment in accordance with an embodiment of the present disclosure.

FIG. 7C illustrates how an SBSE-bead protection apparatus opens and closes in accordance with an embodiment of the present disclosure.

FIG. 7D illustrates an SBSE-bead protection apparatus with a spring mechanism in accordance with an embodiment of the present disclosure.

FIG. 7E illustrates an SBSE-bead protection apparatus with a suspension attachment in accordance with an embodiment of the present disclosure.

### DETAILED DESCRIPTION

The following description is presented to enable any person skilled in the art to make and use the present embodiments, and is provided in the context of a particular application and its requirements. Various modifications to the disclosed embodiments will be readily apparent to those skilled in the art, and the general principles defined herein may be applied to other embodiments and applications without departing from the spirit and scope of the present embodiments. Thus, the present embodiments are not limited to the embodiments shown, but are to be accorded the widest scope consistent with the principles and features disclosed herein.

The data structures and code described in this detailed description are typically stored on a computer-readable storage medium, which may be any device or medium that can store code and/or data for use by a computer system. The computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing computer-readable media now known or later developed.

The methods and processes described in the detailed description section can be embodied as code and/or data, which can be stored in a computer-readable storage medium as described above. When a computer system reads and executes the code and/or data stored on the computer-readable storage medium, the computer system performs the methods and processes embodied as data structures and code and stored within the computer-readable storage medium. Furthermore, the methods and processes described below can be included in hardware modules. For example, the hardware modules can include, but are not limited to, application-specific integrated circuit (ASIC) chips, field-programmable gate arrays (FPGAs), and other programmable-logic devices now known or later developed. When the hardware modules are activated, the hardware modules perform the methods and processes included within the hardware modules.

Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

### Implementation Details

The disclosed embodiments provide a methodology for detecting the HLB pathogen by gathering VOC profiles for plants at different health statuses: healthy (control), infected asymptomatic, mildly infected symptomatic, and severely symptomatic. These VOC profiles are then assessed using a model containing coefficient values for specific biomarkers to determine an infection status for a plant. An in situ sample-collection methodology is described specifically for Hamlin sweet orange (Citrus sinensis L. Osbeck) and Valencia trees, but can also be applied to other C. sinensis or Rutaceae species in general. The methodology can also be adjusted for other crops.

The in situ collection of samples can be carried out using designated SBSE PDMS-based beads (Twisters™, GERSTEL GmbH & Co.KG), although other appropriate sorbent-based sampling methodologies can be used. Note that prior to the analysis by gas chromatography and/or mass spectrometry (GC/MS), the SBSE beads should ideally be conditioned to remove any starting-point adsorbed chemicals from background environmental chemicals, as recommended by the manufacturer. For initial sampling, the beads can be positioned near the surface of the leaf in a stainless steel protective mesh enclosure to protect the beads from dust, pollen or other particulate contamination. To limit effects of the diurnal cycle on leaf VOCs, sampling should ideally be carried out at specific times in the day. The exposure time depends on the efficiency of VOC production by plants and their affinity to the sorbent material. For example, a suggested sampling time is approximately 1-2 hours, depending on foliage thickness. However, a longer or shorter sampling time may also be used; depending on circumstances, a sampling time as short as a minute or a few minutes to multiple hours may be appropriate. An important sampling parameter is the ambient temperature, which ideally occurs in the 60-75° F. range. Samples may also be gathered outside of this temperature range, but may lead to altered volatile output and potentially impaired prediction accuracy. After sampling, the SBSE beads are collected, sealed in glass vials and routed for GC/MS analysis. This process may be automated for mass screening of many trees.

The VOC measurements can be carried out using gas chromatography and/or mass spectrometry (GC/MS). An exemplary GC/MS analysis process for volatile compounds captured by the SBSE methodology is described as follows. This analysis is performed using a 6890 gas chromatograph (Agilent Technologies, Santa Clara, Calif.) equipped with a thermal desorption unit (TDU) (GERSTEL GmbH & Co. KG, Mülheim an der Ruhr, Germany) with a cryo-cooled injection system inlet (CIS4) (GERSTEL GmbH & Co. KG), and interfaced to a Pegasus IV time-of-flight mass spectrometer (LECO, St. Joseph, Mich.). However, note that other equivalent chemical analysis platforms can be used. Volatiles that are trapped using Twisters are thermally desorbed in the TDU in splitless mode. The desorbed analytes are then cryofocused in the CIS4 inlet with liquid nitrogen (−120° C.), heated from −120° C. to 260° C., and analyzed on an Rtx-5SilMS column with a 10 m integrated guard column (95% dimethyl/5% diphenyl polysiloxane film; 30 m×0.25 mm (inside diameter)×0.25 μm d_{f} (Restek Corporation, Bellefonte, Pa.)). During this process, the GC oven temperature program proceeds as follows: initial temperature of 45° C. with a 2 minute hold, followed by a 20° C./min ramp up to 300° C. with a two-minute hold, and thereafter a 20° C./min ramp up to 330° C. with a 0.5-minute hold with a constant 1 mL/min flow of the carrier gas (99.9% He). Mass spectra are then acquired at 25 spectra/sec with a mass range of 35-500 m/z, with the detector voltage set at 1800 V and the ionization energy at 70 eV. Raw GC/MS data can then be pre-processed by Leco ChromaTOF software, or any similar software, to extract individual peaks from the resultant chromatogram. The compounds are identified based on similarity of mass spectra and retention indices to that of corresponding chemical standards. The list of compounds and their corresponding abundances are generated for each sample. In the generated tables of compounds, every peak can be normalized using conventional techniques, such as against an internal standard. Other normalization operations can be performed against a stable biogenic chemical abundance measured from the sample, or against a stable ambient standard at the point of sampling. The instrumentation used for GC/MS analysis can also be varied. Although alteration of compound coverage due to instrument-specific differences is expected (changes in limits of detection for various compounds, discrimination against some compounds), the application of the present approach is still possible, albeit with potentially diminished robustness.

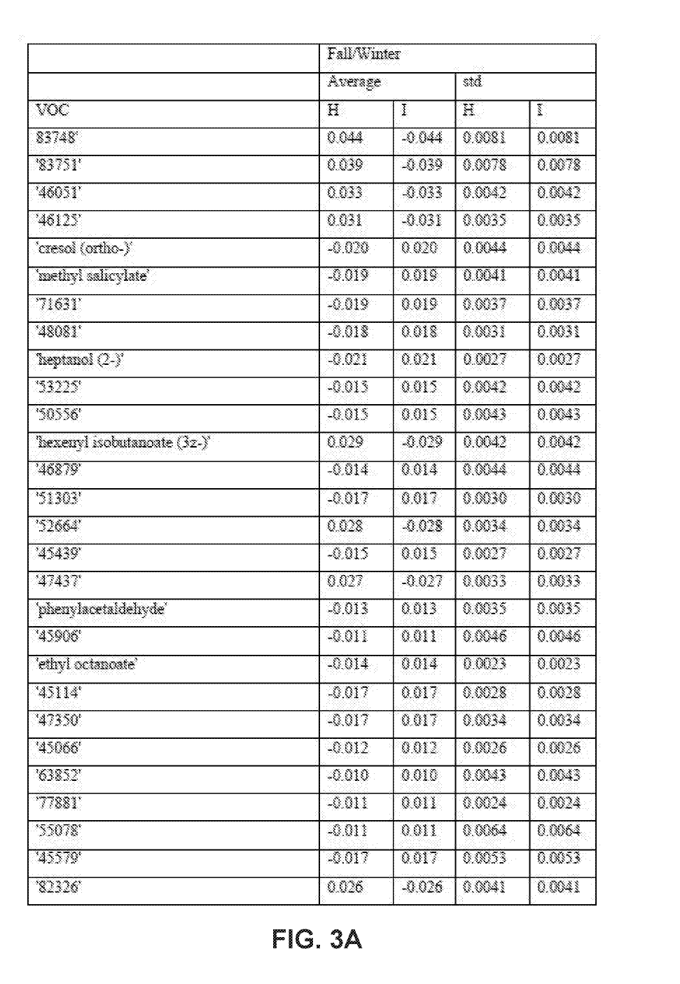

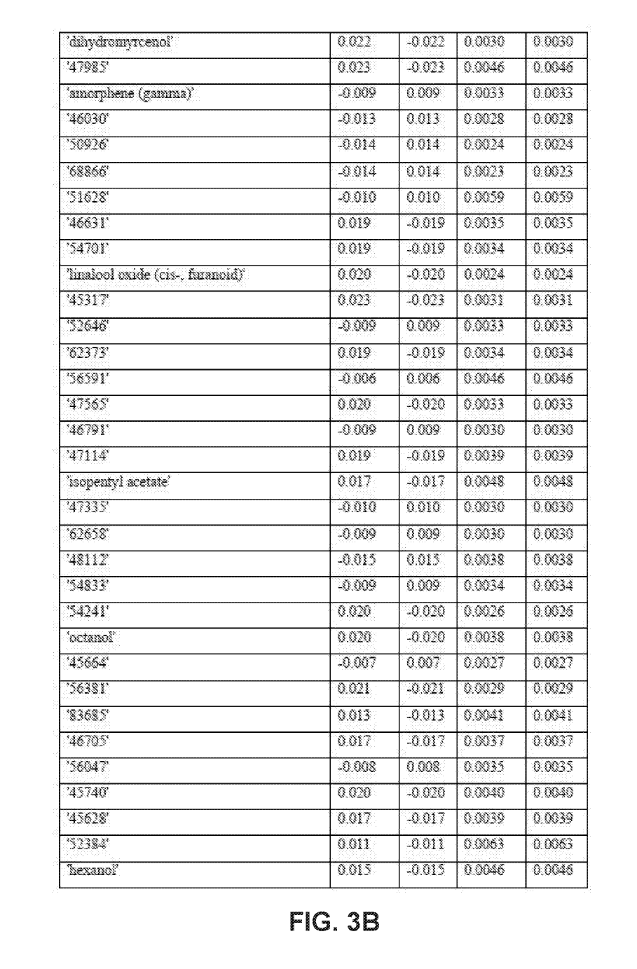

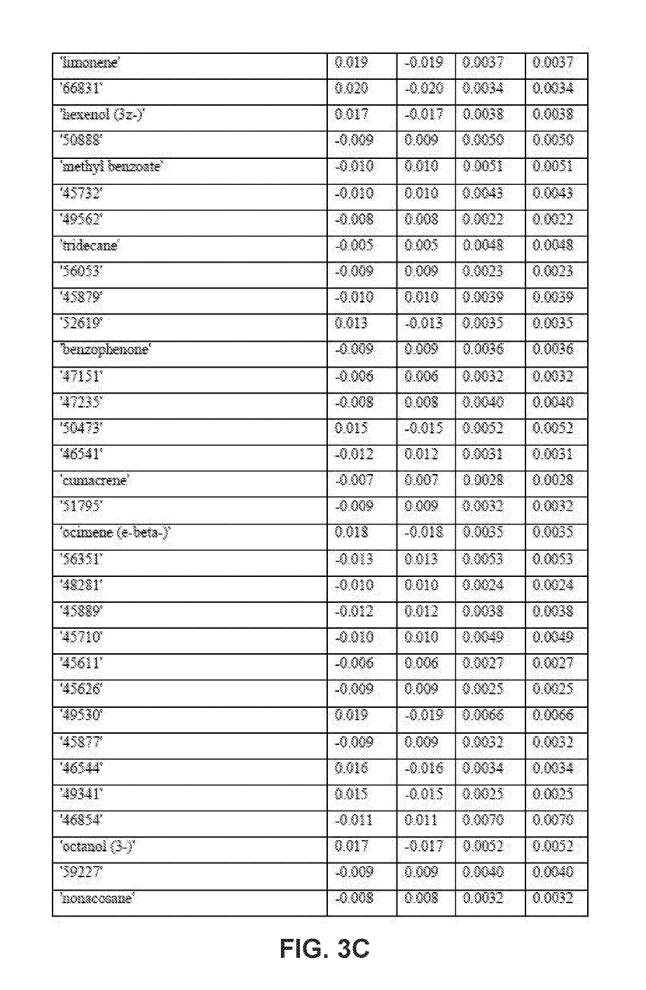

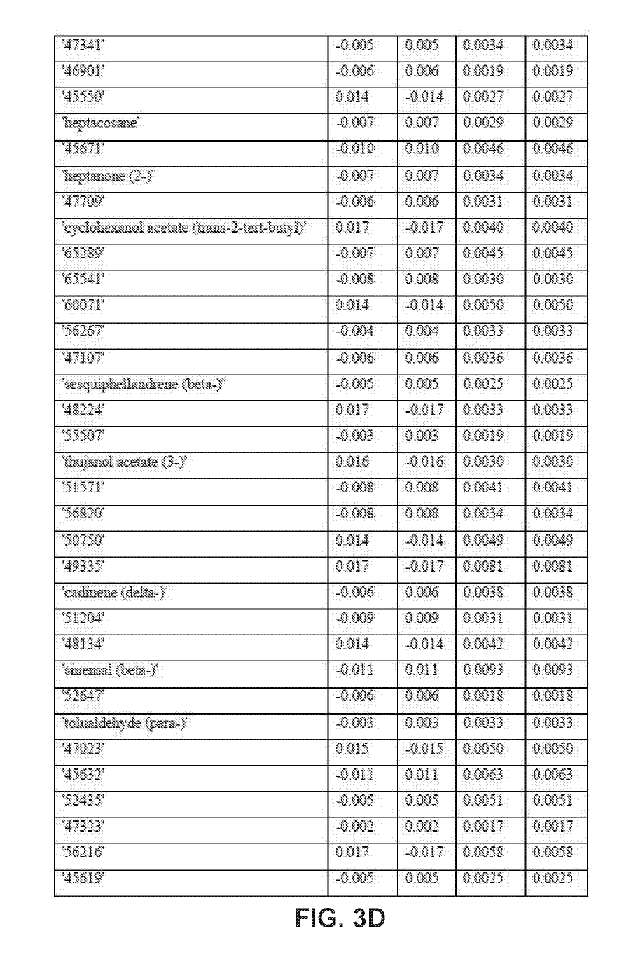

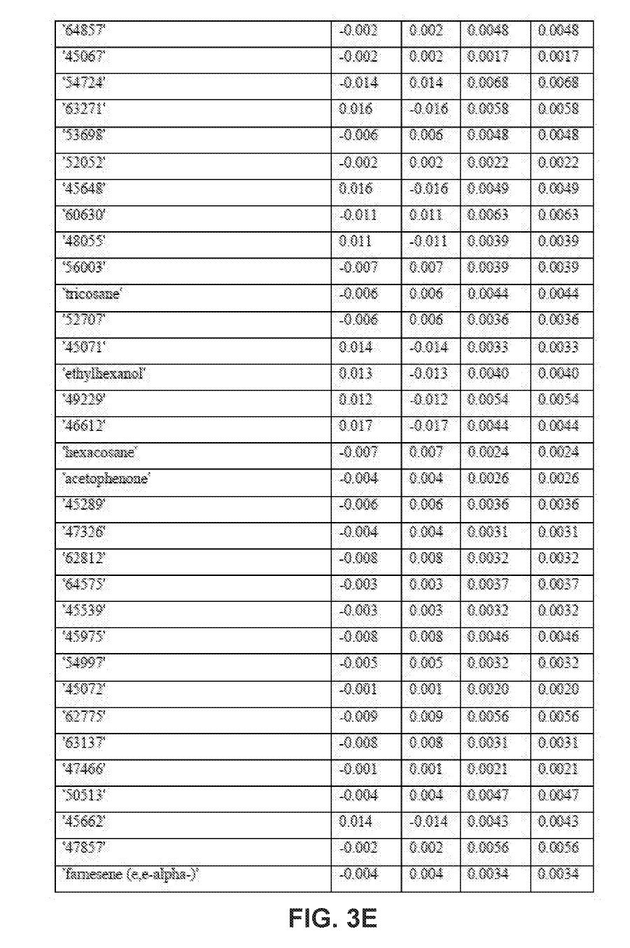

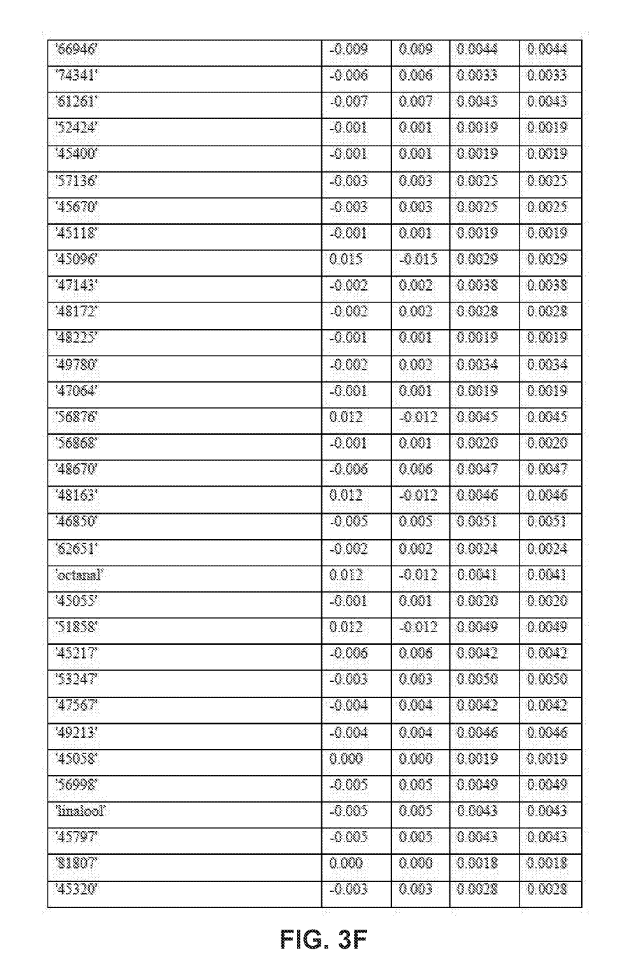

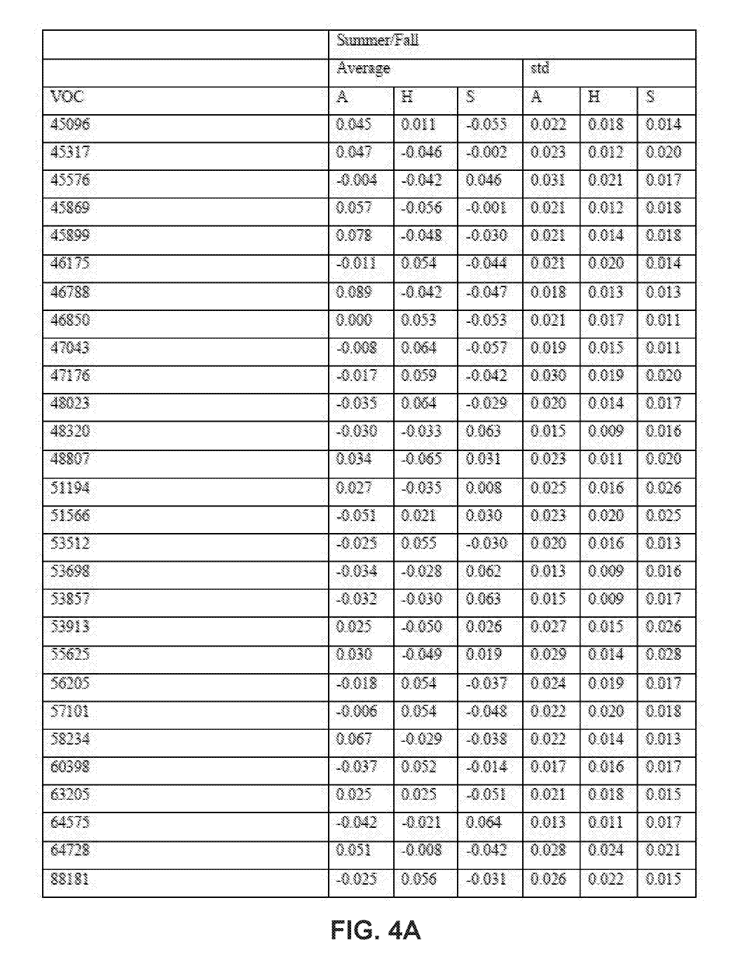

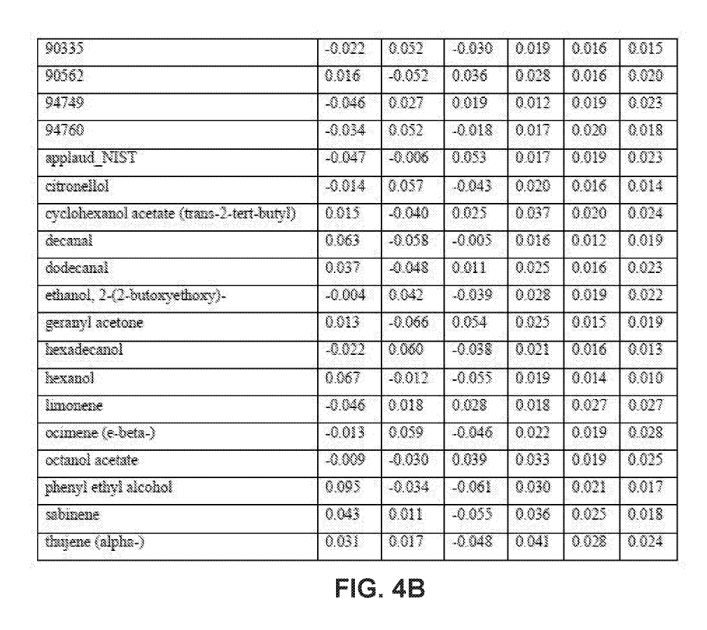

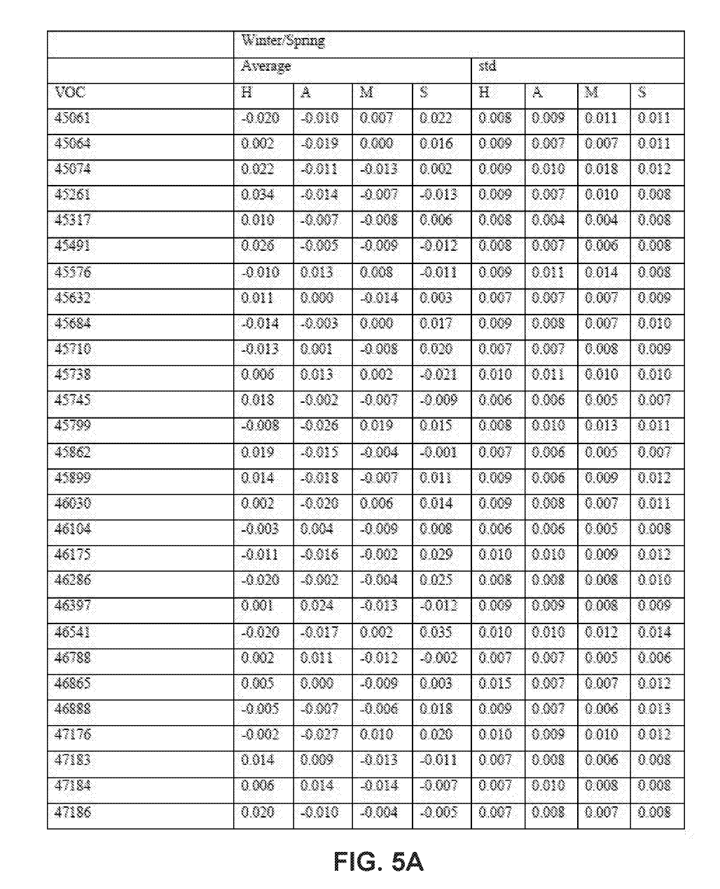

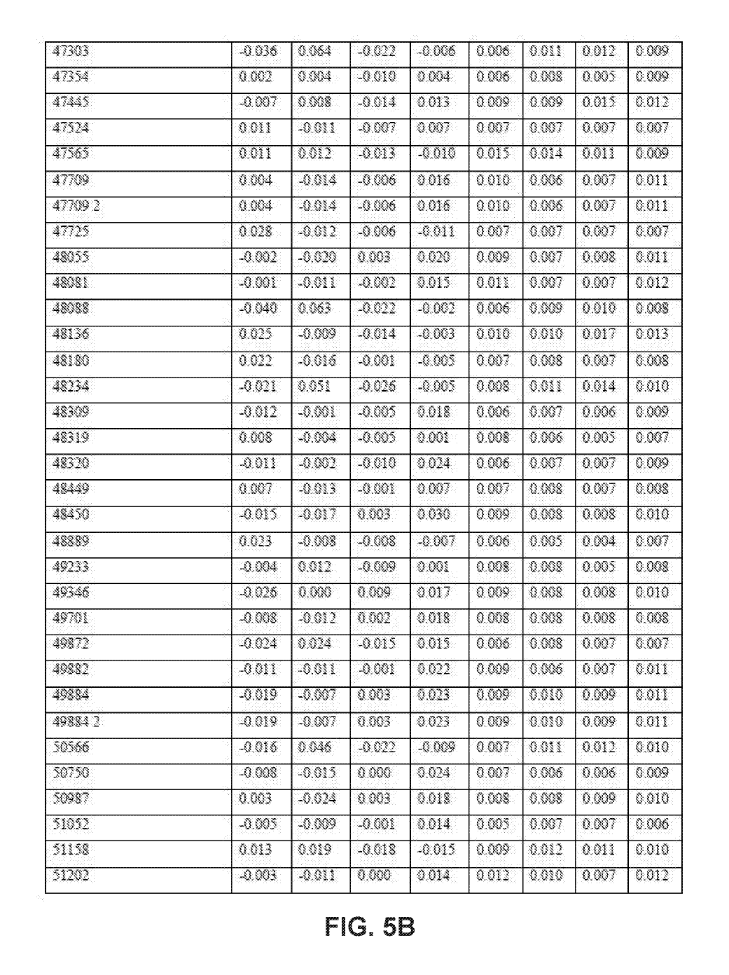

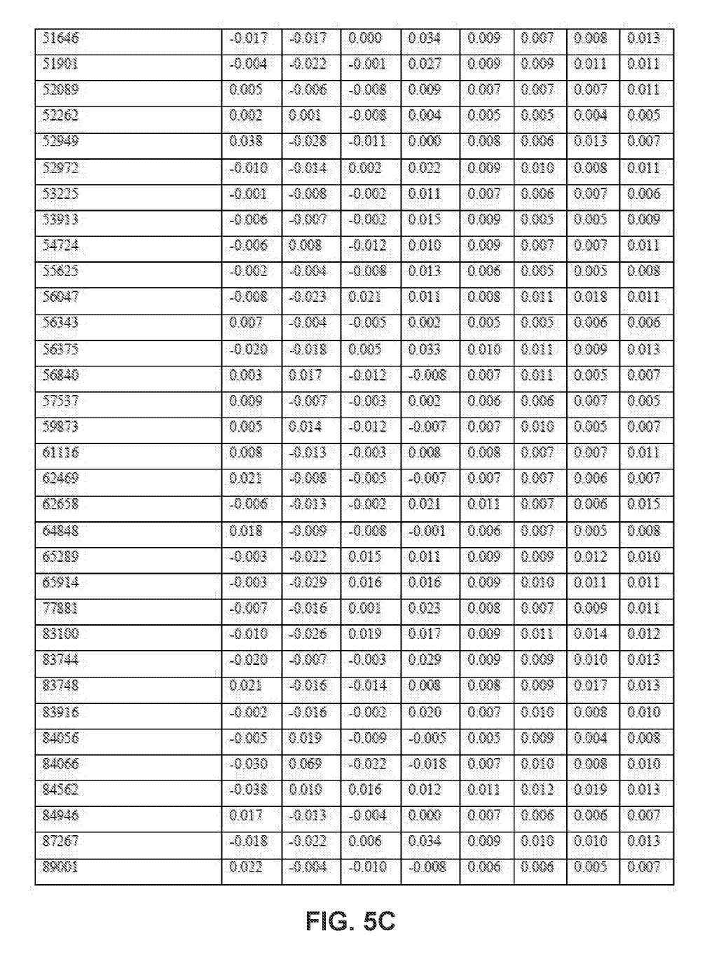

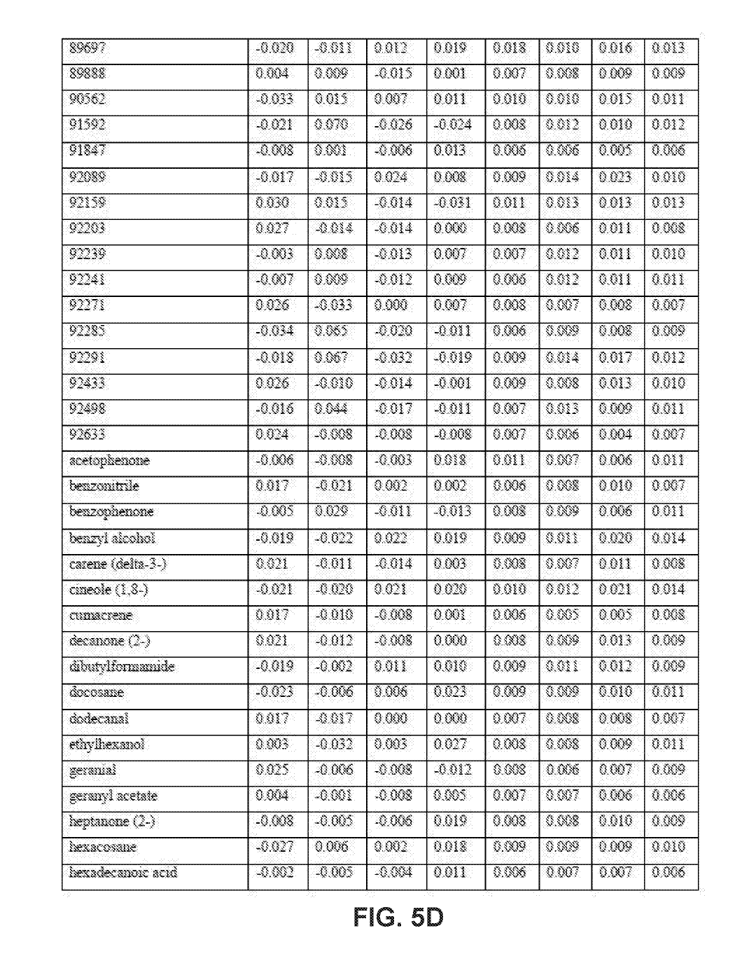

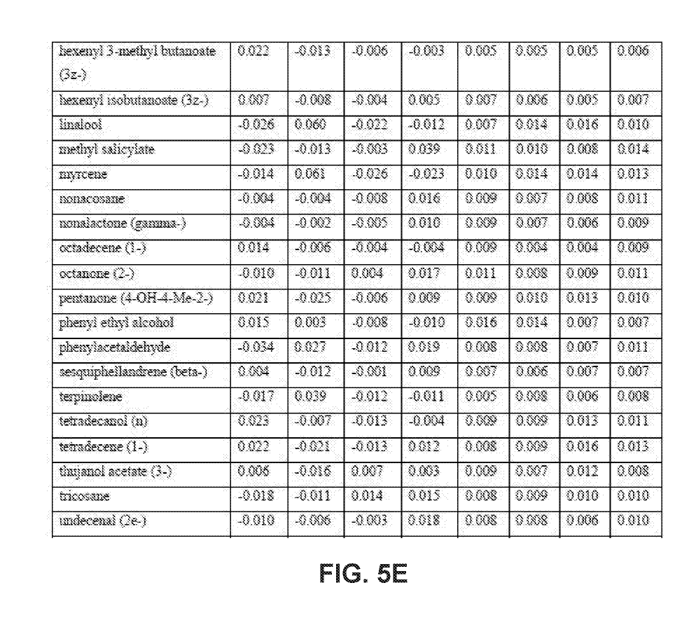

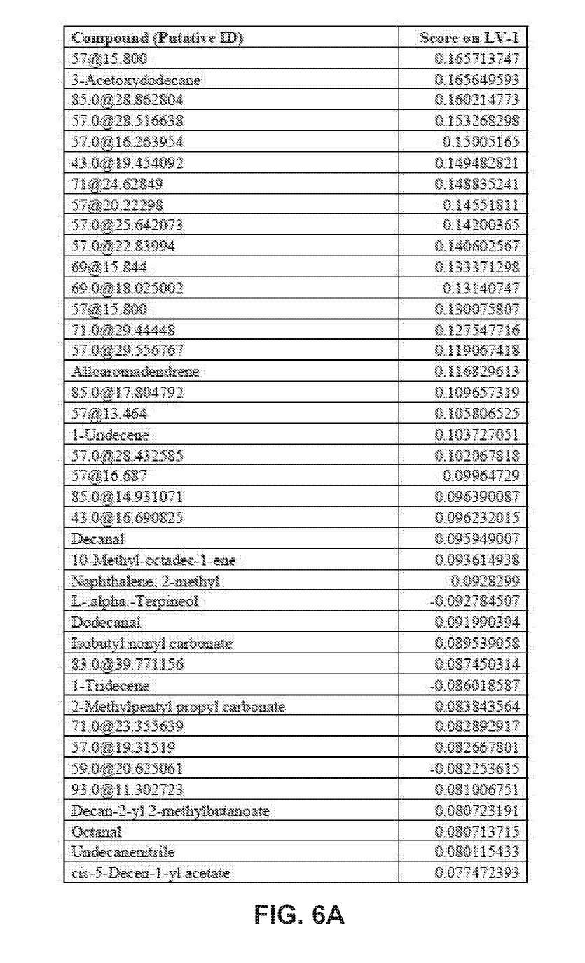

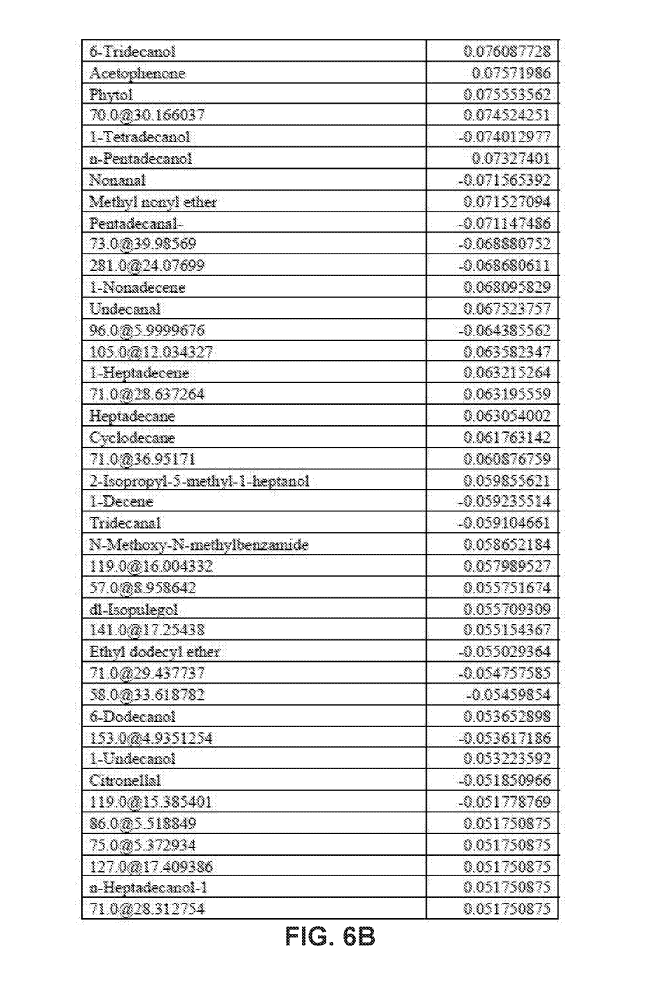

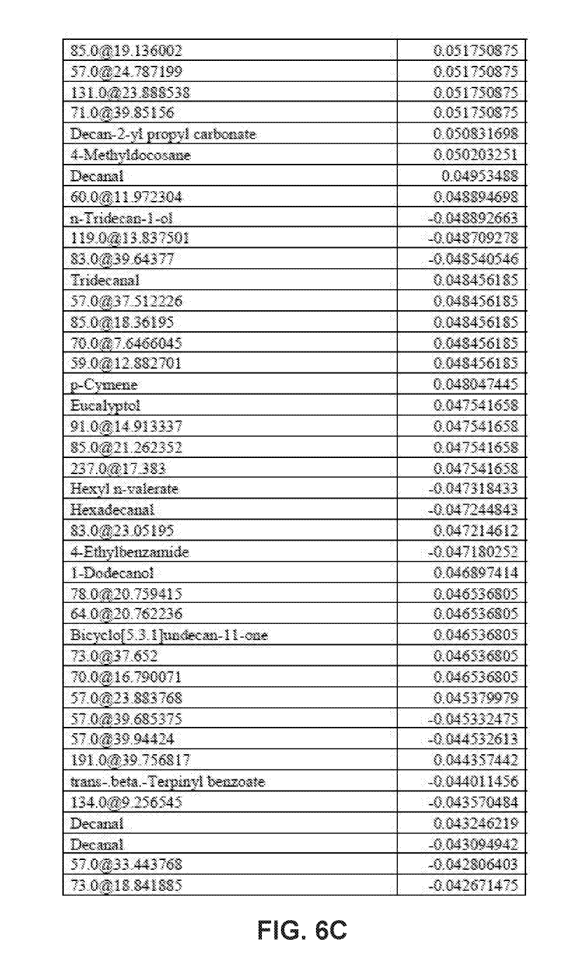

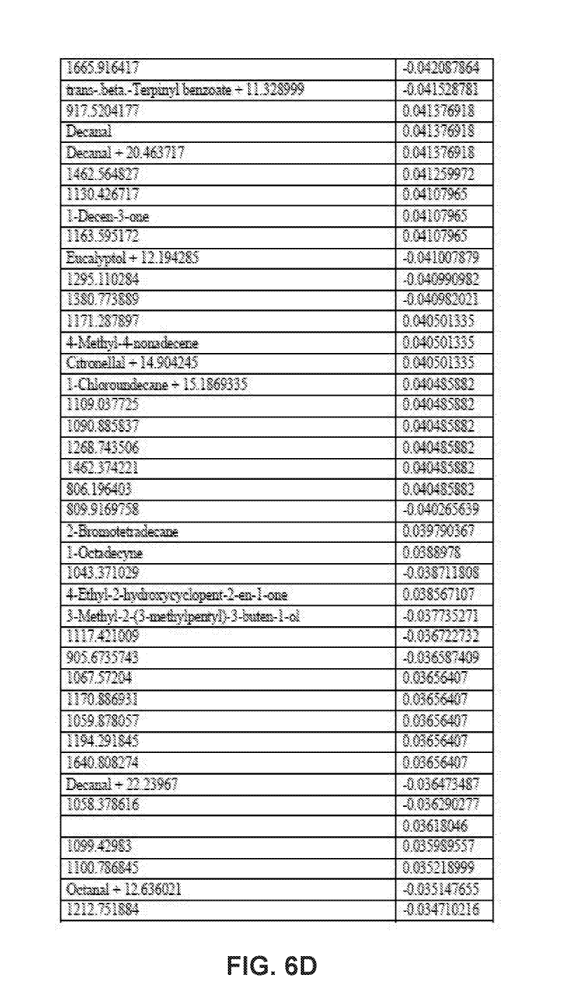

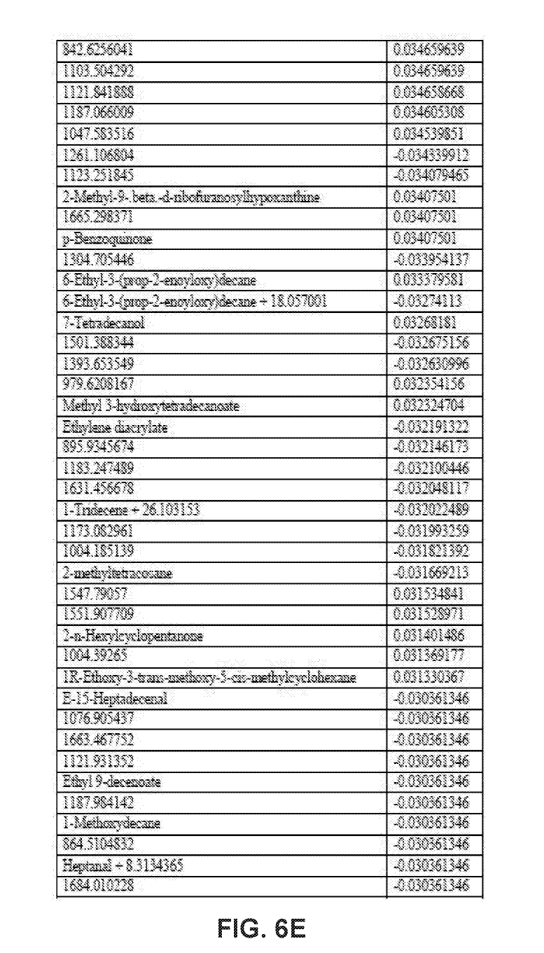

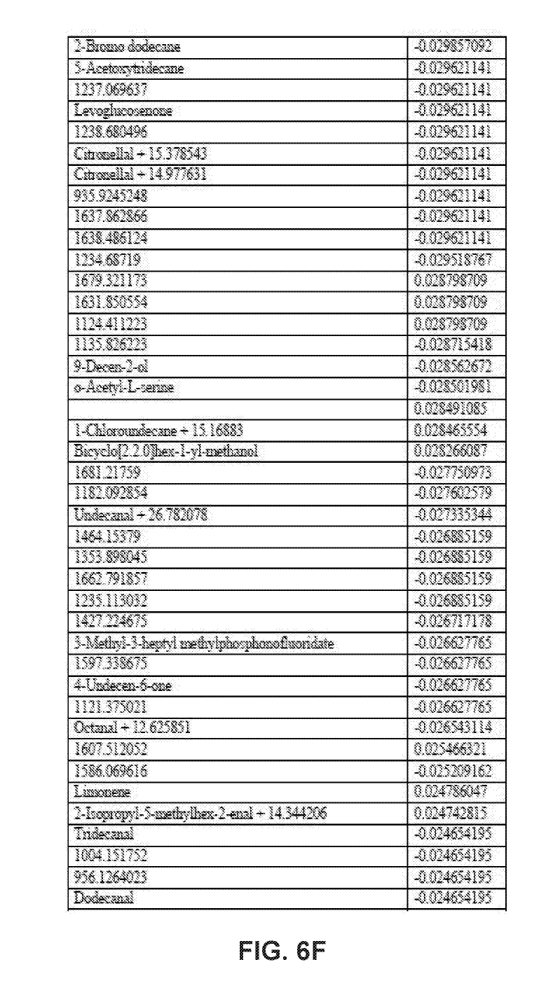

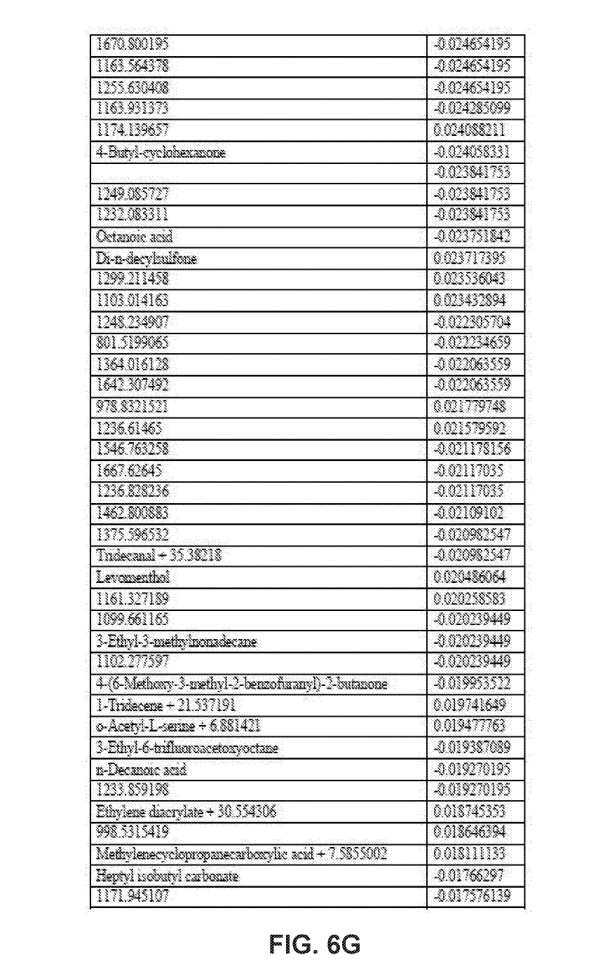

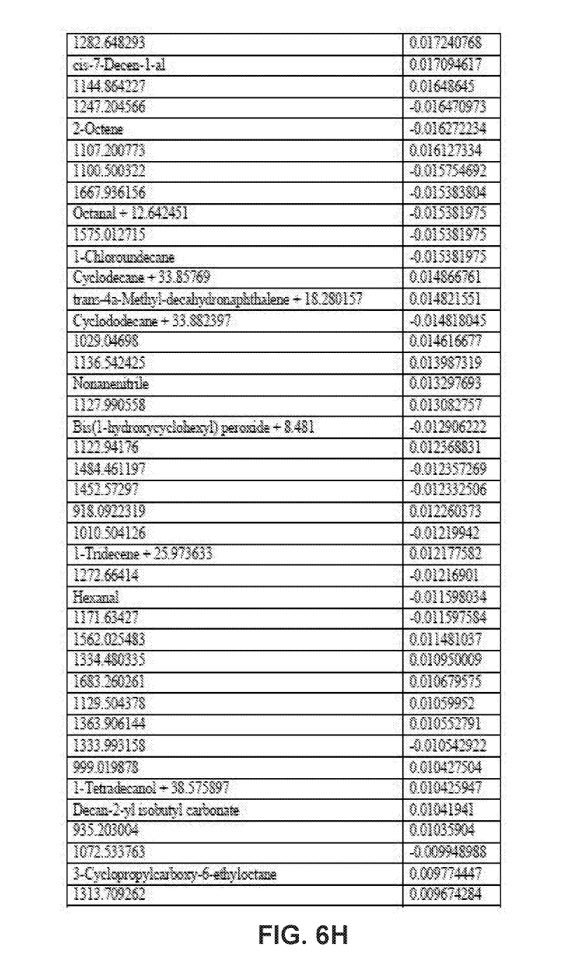

A statistical model can be applied depending on the season to the unknown samples to generate probability values for the infection status. Other closely related statistical methods such as PLS-based tools, linear regression methods or ensemble learning methods like random forests can also be used. These probability values are obtained by multiplying the intensity of peak values for each compound (these values are auto-scaled) by its corresponding coefficient given in one or more of the tables of season-specific coefficients for determination of HLB infection health status that appear in FIGS. 3A-3F, 4A-4B, 5A-5E and 6A-6I. (Note that, within these tables, the numbered compounds refer to entries in the Fiehn volatile database, which can be found at http://fiehnlab.ucdavis/edu/). However, other databases can be used. The values in these tables relate to the categorizations assigned during the generation of the model and correspond to a probability of accuracy for each category (healthy versus infected with different symptom severity). The resulting value represents the probability of infection status. When the probability value corresponding to healthy exceeds 0.7 and the values within the other categories are below 0.4, the sample should be categorized as coming from a healthy tree. The same criteria can be applied for infected trees. For example, if the value obtained falls between 0.7-0.4, the sample should be considered undetermined (suspicious). Note that these ranges of sample values are merely exemplary ranges, and these ranges can be changed based on new data.

The database of disease-specific biomarkers can be expanded to include a variety of pathogens, and the database can be further updated when new biomarkers are discovered. Also, compounds that are found to result in insufficiently robust differentiation can be removed from the database. The chemical compounds specific to Hamlin and Valencia orange trees affected by HLB disease include the compounds listed in the tables that appear in FIGS. 3A-3F, 4A-4B, 5A-5E and 6A-6I. However, the seasonality of biomarker changes can be further assessed with increased time resolution.

The application of this methodology to citrus other than sweet orange is feasible, but a decrease in accuracy may result. For example, in a variation of the methodology that uses a GC single quadrupole MS instead of the above-described GC TOF MS (with corresponding differences in GC protocols) for testing of grapefruit, only a subset of biomarkers overlap, wherein the subset includes, but is not limited to: octanal, limonene, 2-ethyl-1-hexanol or isomer, and acetophenone.

### Chemical-Analysis System

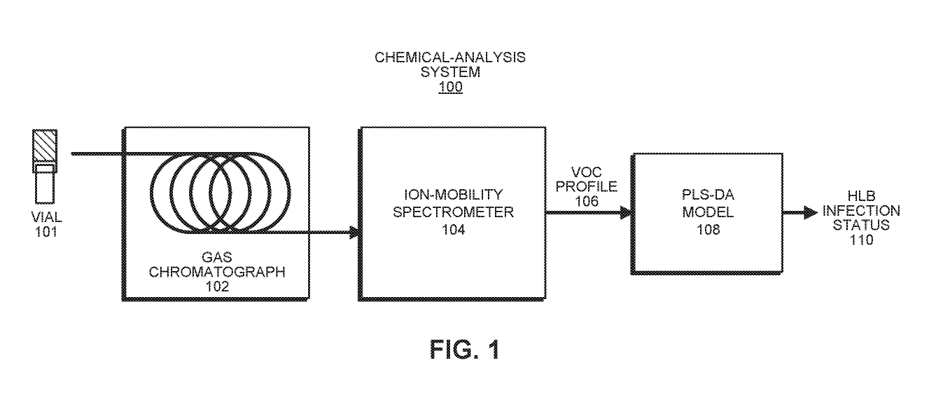

In summary, FIG. 1 illustrates a chemical-analysis system 100 in accordance with the disclosed embodiments. As mentioned above, this chemical-analysis system 100 receives a vial 101 containing a sample in the form of a sorbent bead, which has been exposed to VOCs emitted by a citrus plant. The VOCs are desorbed from the bead and fed into an injection port of gas chromatograph 102, which subjects the VOCs to a specific oven temperature profile. Next, the VOCs are fed into a sensor that comprises an ion-mobility spectrometer 104, which produces a set of measured values for the VOCs that collectively form a VOC profile 106 for the sample. (Note that it is possible to use one or more other types of sensors, such as a mass spectrometer.) Finally, the VOC profile 106 is processed using PLS-DA model 108, which determines an HLB infection status 110 for the citrus plant.

### Method of Operation

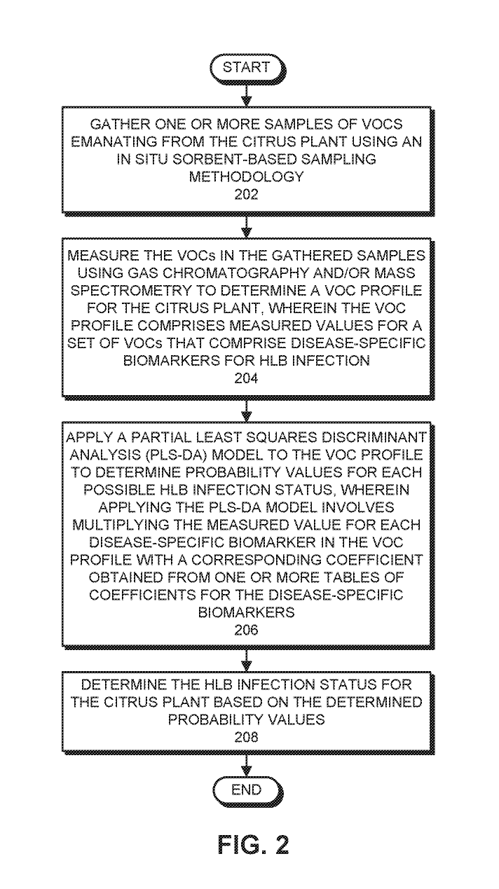

FIG. 2 presents a flowchart illustrating how chemical-analysis system 100 operates in accordance with an embodiment of the present disclosure. First, the system gathers one or more samples of VOCs emanating from the citrus plant using an in situ sorbent-based sampling methodology (step 202). Next, the system measures VOCs in the gathered samples using gas chromatography and/or mass spectrometry to determine a VOC profile for the citrus plant, wherein the VOC profile comprises measured values for a set of VOCs that comprise disease-specific biomarkers for HLB infection (step 204). Then, the system applies a partial least squares discriminant analysis (PLS-DA) model to the VOC profile to determine probability values for each possible HLB infection status, wherein applying the PLS-DA model involves multiplying the measured value for each disease-specific biomarker in the VOC profile with a corresponding coefficient obtained from one or more tables of coefficients for the disease-specific biomarkers (step 206). Finally, the system determines the HLB infection status for the citrus plant based on the determined probability values (step 208).

### Apparatus for Handling Sorptive Substrates

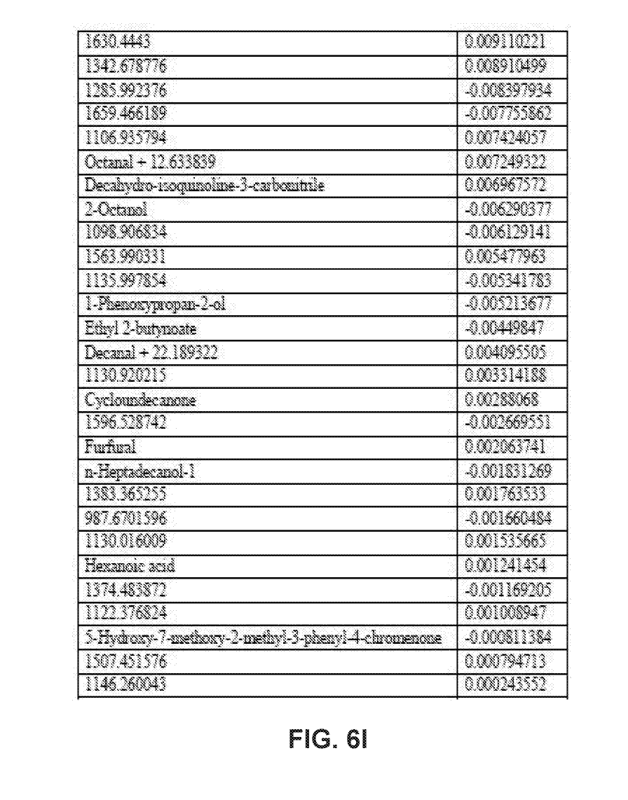

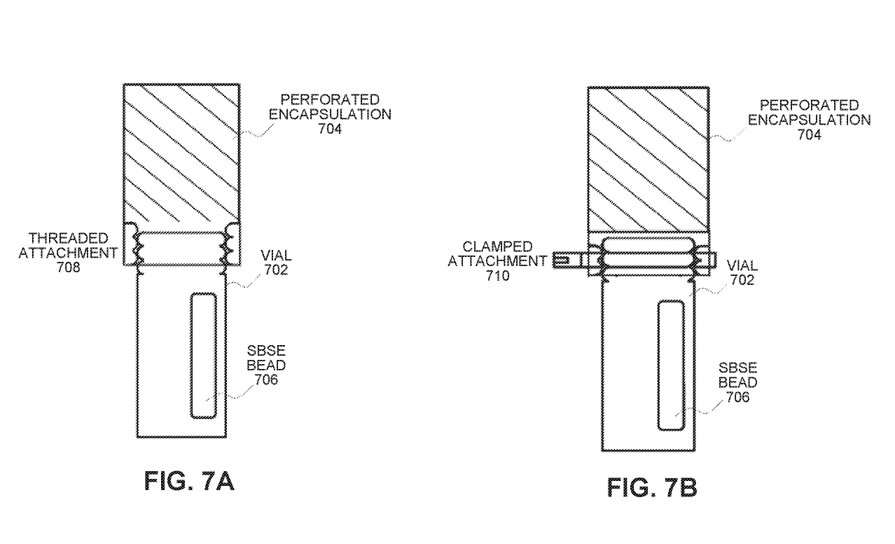

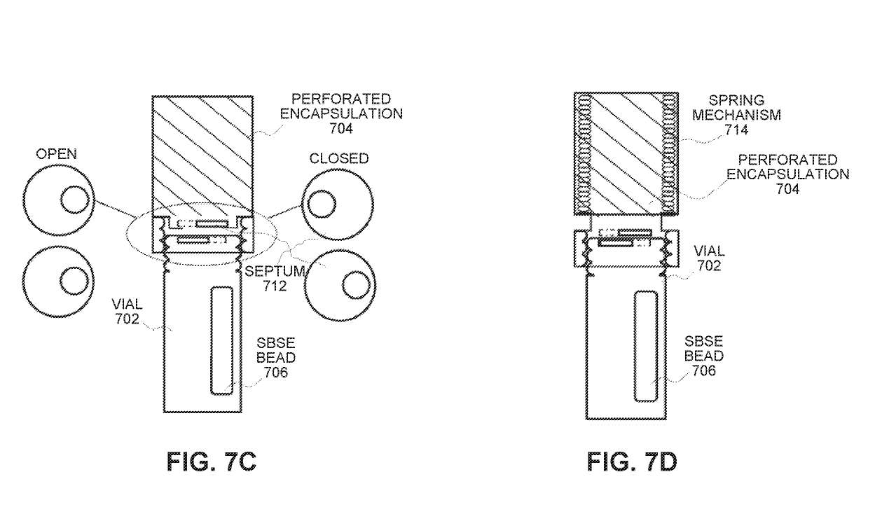

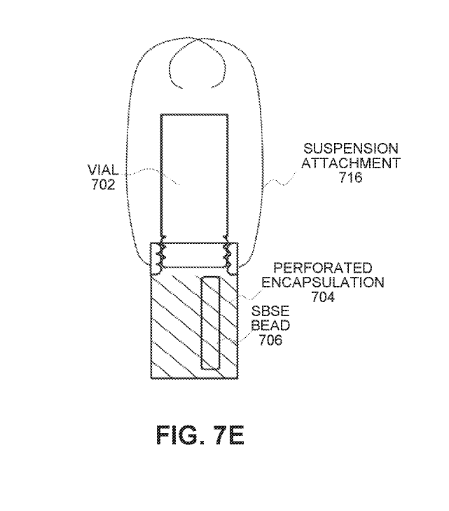

The present invention also relates to an apparatus that facilitates easier manipulation of polydimethylsiloxane (PDMS)-based stir bar sorptive extraction (SBSE) beads as they are used to collect VOCs emitted by plants or other volatiles sources for later gas chromatography/mass spectrometry (GC/MS) analysis. (However, note that other analytical chemistry analysis techniques can be used, such as FTIR, FAIMS/DMS, DT IMS, or discrete chemical sensors tuned for a specific VOC of interest.) Referring to FIG. 7A, this apparatus provides an all-in-one device, which enables an SBSE bead 706 (or other packaged or pre-formed sorbent) to be easily manipulated and transported inside a sealed vial 702 from the laboratory to a plant, a grove or a post-harvest location. The apparatus can then be hung inside the plant foliage or in close proximity to an agricultural product while providing a perforated encapsulation 704 comprising a protective inert mesh (e.g., stainless steel or PTFE). This inert mesh prevents the SBSE bead 706 or sorbent material from becoming contaminated by dust, pollen, tree sap, or other contaminants, and allows the bead 706 or sorbent material to be easily resealed inside the same device where it may be transported back to the laboratory for GC/MS analysis while minimizing manual handling.

FIGS. 7C and 7D illustrate an SBSE protective apparatus, which is amenable to shipping and handling. The apparatus can be used to deploy an SBSE bead by transfer through a septum 712 by using gravity as in FIG. 7C or by using spring force provided by a spring mechanism 714 as is illustrated in FIG. 7D. The apparatus may remain attached to the vial after sample collection and during shipping and handling. However, it needs to be removed for cleaning prior to GC/MS analysis.

In an exemplary use case, the SBSE-protective apparatus is deployed in situations where abundances of specific compounds need to be measured in situ. The protective apparatus is interfaced with a brown vial containing an SBSE bead supplied by GERSTEL GmbH & Co. KG, Mülheim an der Ruhr, Germany. (Note that the plastic screw top cap needs to be removed prior to the interfacing.) The apparatus is then engaged to transfer the SBSE bead from the vial into the mesh enclosure without physically removing the bead from its containing vial to transfer it. This eliminates the possibility of exposing the bead to contamination associated with differences in handling that may affect the retained compounds or may potentially cause damage. The apparatus is then placed within foliage as appropriate for the sample collection during an exposure time window. The bead remains exposed to the volatiles emitted by the plant as the volatiles penetrate the mesh due to normal gas exchange.

After sample collection, the apparatus is removed from the tree and the SBSE bead is transferred back into its vial without physical removal from the apparatus. Note that versions of the apparatus that remain on the vial after collection (or are removed after collection) are possible. While retention of the apparatus reduces the amount of physical manipulation of the beads, this type of design may interfere with the GC/MS sampler. Also, the apparatus will eventually get contaminated during the repeated use and shipping/handling. Note that the removable designs illustrated in FIGS. 7A-7B provide a threaded attachment 708 and a clamped attachment 710, respectively, which facilitate cleaning and reuse of the apparatus between deployments, although an additional handling step of cap removal/replacement between apparatus placement/removal steps is required. Also note that an automated power tool, which is specifically designed to screw/unscrew the cap and place the apparatus, can be used to facilitate this process.

In another use case, which is unrelated to in situ testing of volatiles from plants, the technique can be used to test for volatiles in different environments. Examples of such environments include: storage warehouses, such as those where various kinds of produce are stored; food manufacturing processing facilities; and meat packing plants. Similarly to the first use case, the apparatus, which is interfaced to the SBSE bead-containing vial, is engaged and the bead is deployed by transferring it into the mesh compartment without removing the bead. This type of deployment technique prevents potential contamination, especially in the above-listed environments, as well as reducing the manual handling of the bead.

In addition to gas-phase sampling applications, liquid solution testing applications are also possible. The mesh compartment of the apparatus containing the SBSE bead can be immersed into vats of the liquids that require testing. However, the vial needs to remain above the liquid to prevent contamination. When testing is complete, the bead is retracted into the vial and the vials are handled in the same way as for volatiles testing.

In another use case, the apparatus could be equipped with a tracking device such as a laser tag, barcode, or GPS device. The cost and/or functionality of this tracking feature may be tailored to the requirements of specific applications. An example would be tracking and logging the samples that are collected from sites of infection that require high scrutiny and close tracking of the sample. The GPS position, if and when required, can be also used for sample collection location identification, as well as detailed tracking of the sensitive samples or those that require additional security during collection and transportation. The type of tagging system facilitates a sampling process flow that is trackable for auditing purposes. This is especially beneficial for regulatory uses as well as security applications.

In another use case, a labeling feature can be added to the apparatus. For example, a low-cost feature, such as a barcode, could be printed on the apparatus itself, or could be removable or replaceable for specific applications. This barcode information can be used to track sample origin, sample location, and the status of the apparatus itself (whether it needs to be cleaned/refurbished) as well as the GC/MS or other chemical-analysis status. This information can be compiled in a database to facilitate: designing and optimizing the business process flow; generating reports as needed; and managing the protective apparatus life cycle.

Note that plants can experience multiple physiological and biochemical responses when exposed to pathogens or injury. For instance, citrus trees infected with Candidatus Liberibacter asiaticus (CLas) bacteria exhibit altered gene expression, and the host response to infection appears to modify certain metabolic signatures of the trees. Hence, it is no surprise then that an affliction-initiated cascade of biological change can alter the end products of certain metabolic pathways, such as the VOCs emitted by the trees. All living organisms naturally produce VOCs, and these chemicals have been shown to be closely associated with plant health. One such example is the ability to differentiate VOC profiles of healthy citrus from those of CLas-infected citrus in field Hamlin sweet orange (Citrus sinensis L. Osbeck) using in situ VOC collection and detection methods.

One such diagnostic method uses PDMS-based SBSE beads (or other sorbent phases) combined with a bench-top GC/MS instrument. In addition to preconcentration of low-concentration analytes from (predominantly) aqueous solutions, beads can be used for volatiles preconcentration out of gas phase. In most VOC investigations using SBSE-GC/MS, volatiles are first preconcentrated from the SBSE bead and then introduced to the gas chromatograph. In a controlled environment such as a laboratory, this process can be streamlined and the risk of contamination during the volatile sampling process can be relatively low.

However, collecting plant VOCs in situ, for example from plants found in fields, groves or postharvest locations, introduces challenges during the volatile collection process. First, the sorbent must be transported from the laboratory to the plant in an extremely inert environment (usually a clean, sealed vial) to prevent contamination during the transportation process. Then, the sorbent must be hung within the foliage of the plant, or presented near a postharvest source of VOCs. Note that fields and groves are extremely dynamic environments, increasing the risk of the sorbent coming in contact with dust, pollen, tree oils, insects, and contaminants. Thus, protective stainless steel mesh cages are sometimes used in the form of commercially available beverage strainers. The SBSE bead is removed from its clean vial into the strainer and hung in the tree foliage for a certain amount of time. Referring to FIG. 7E, this hanging operation is facilitated by a suspension attachment 716 coupled to the apparatus. After the VOC collection is complete, the SBSE bead must be resealed inside the vial to be transported back to the laboratory. At every point in this process, where the SBSE bead is manually manipulated (moved from vial to cage and back again), the user runs the risk of contaminating the SBSE bead, misplacing it in the wrong tree or vial or physically damaging the bead, for example, by dropping the bead or accidentally squeezing the glass too hard. Note that these beads are normally handled with laboratory tweezers, but it is easy to drop a bead onto the ground where it will be contaminated, or worse the bead may roll away or be lost in the soil and never recovered. There is also the potential for mislabeling and mismatching the sample numbers resulting in experimental results being attributed to the wrong tree.

The disclosed apparatus streamlines the VOC-sampling process with SBSE beads or other sorbents, and greatly reduces the possibility of contamination or mislabeling. As mentioned above, the apparatus comprises a storage vial, such as those in which beads are supplied by a manufacturer and a perforated cap attachment, both of which have the capacity to hold at least one SBSE bead. The vial can be composed of an inert and airtight material ensuring the SBSE bead will not interact with the environment while it is being transported or stored. Furthermore, the vial material may be designed to be opaque and to withstand various temperatures; the vial material can be otherwise tailored for specific application requirements. A chemically inert perforated material is included to interface with the vial to enable the SBSE bead to be captured as it is released from the vial, without any special handling or contact with the SBSE bead, and also to allow the bead to be held within the perforated material. The perforated material may include a mechanism to facilitate separation and reattachment to the vial, and also a mechanism to facilitate suspension in diverse environments. The perforated material allows the SBSE bead to adsorb the environmental VOCs without significantly denaturing the sorbent-sample interaction or introducing any extraneous chemical components. The perforated surface may also be treated to further improve inertness to select chemicals. Furthermore, the entire device can be easily cleaned at temperatures around or above 160° C. or in a solvent wash to remove any lingering volatile compounds or compounds that may result in production of volatiles so that the device can be reused for volatile sampling multiple times. These devices can also be uniquely numbered and tracked to provide a seamless process flow of analysis from lab to field, and back to lab, which facilitates reduced risk of mislabeling samples.

Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

The foregoing descriptions of embodiments have been presented for purposes of illustration and description only. They are not intended to be exhaustive or to limit the present description to the forms disclosed. Accordingly, many modifications and variations will be apparent to practitioners skilled in the art. Additionally, the above disclosure is not intended to limit the present description. The scope of the present description is defined by the appended claims.

What is claimed is:

1. A method for detecting Huanglongbing (HLB) infection in a citrus plant, comprising:
gathering one or more samples of volatile organic compounds (VOCs) emanating from the citrus plant;
measuring the VOCs in the gathered samples to determine a VOC profile for the citrus plant, wherein the VOC profile comprises measured values for a set of VOCs that comprise disease-specific biomarkers for HLB infection; and
determining an HLB infection status for the citrus plant by analyzing the VOC profile.

2. The method of claim 1, wherein gathering the samples of the VOCs involves in situ collection of the samples.

3. The method of claim 2, wherein the in situ collection of the sample involves using a sorbent-based sampling methodology.

4. The method of claim 3, wherein the sorbent-based sampling methodology involves using a polydimethylsiloxane (PDMS)-based absorptive bead.

5. The method of claim 1, wherein gathering the samples involves gathering each sample for a predetermined duration spanning minutes to hours.

6. The method of claim 1, wherein gathering the samples involves gathering the samples at specific times of day.

7. The method of claim 1, wherein measuring the VOCs in the gathered samples involves using gas chromatography and/or mass spectrometry to perform the measurements.

8. The method of claim 1, wherein determining the HLB infection status for the citrus plant involves:
applying a partial least squares discriminant analysis (PLS-DA) model to the VOC profile to determine probability values for each possible HLB infection status; and
determining the HLB infection status for the citrus plant based on the determined probability values.

9. The method of claim 8, wherein applying the PLS-DA model to the VOC profile involves multiplying the measured value for each disease-specific biomarker in the VOC profile with a corresponding coefficient obtained from one or more tables of coefficients for the disease-specific biomarkers.

10. The method of claim 9, wherein the one or more tables of coefficients account for one or more of: season-specific alterations, varietal alterations, and geographic alterations of the disease-specific biomarkers.

11. The method of claim 9, wherein the one or more tables of coefficients are stored in a database.

12. The method of claim 1, wherein the HLB infection status for the plant comprises at least one of the following:
healthy;
infected asymptomatic;
mildly infected symptomatic; and
severely symptomatic.

13. A device that facilitates handling a sorbent bead to facilitate using the sorbent bead to sample chemical compounds, comprising:
a storage enclosure for holding the sorbent bead, wherein the storage enclosure is sealable to prevent contamination of the sorbent bead during transport and storage;
a sampling enclosure for holding the sorbent bead, wherein the sampling enclosure is perforated to allow chemical compounds to come into contact with the sorbent bead while the sorbent bead is being used to sample the chemical compounds; and
a sealable interface between the storage enclosure and the sampling enclosure, wherein when unsealed, the sealable interface provides an opening to facilitate moving the sorbent bead between the storage enclosure and the sampling enclosure without physical handling of the sorbent bead by a user.

14. The device of claim 13, wherein the sampling enclosure comprises a chemically inert mesh.

15. The device of claim 13, wherein the storage enclosure comprises a sealable vial.

16. The device of claim 13, wherein the storage enclosure is detachable from the device.

17. The device of claim 13, wherein the sampling enclosure is detachable from the device.

18. The device of claim 13, wherein the device further comprises a suspension mechanism for suspending the device at a sampling location while the sorbent bead is held in the sampling enclosure.

19. The device of claim 13, wherein the sorbent bead comprises a stir bar sorptive extraction (SBSE) bead.

20. The device of claim 13, wherein the device includes a tracking mechanism.

21. The device of claim 20, wherein the tracking mechanism comprises a label.

22. The device of claim 21, wherein the label comprises a barcode.

23. The device of claim 21, wherein the label comprises a radio-frequency identification (RFID) tag.

24. The device of claim 20, wherein the tracking mechanism comprises a global-positioning system (GPS) tag.
